(19) United States
(12) Patent Application Publication
(10) Pub. No.: US 2025/0228508 A1
(43) Pub. Date: Jul. 17, 2025

(54) SYSTEM AND METHOD FOR OPTIMAL COMBINING OF COMPUTED TOMOGRAPHY AND POSITRON EMISSION TOMOGRAPHY IMAGING FOR ATTENUATED CORRECTION AND IMPROVED DIAGNOSIS

(71) Applicant: GE Precision Healthcare LLC, Waukesha, WI (US)

(72) Inventor: Gil Kovalski, Haifa (IL)

(21) Appl. No.: 18/410,537

(22) Filed: Jan. 11, 2024

(51) Int. Cl. A61B 6/03; A61B 6/00

(52) U.S. Cl. CPC A61B 6/037; A61B 6/5235; A61B 6/527; A61B 6/5294

(57) ABSTRACT

A method includes reconstructing a computed tomography image of a subject based on computed tomography scan data acquired at a determined respiratory phase with a computed tomography scanner. The method includes generating an attenuation map from the CT image and obtaining positron emission tomography scan data of the subject acquired over multiple respiratory cycles with a positron emission tomography scanner. The method includes gating the positron emission tomography scan data into different respiratory phases and reconstructing a positron emission tomography image utilizing only the positron emission tomography scan data acquired at a same respiratory phase as the determined respiratory phase and the attenuation map from the CT image.

### BACKGROUND

The subject matter disclosed herein relates to medical imaging and, more particularly, to a system and method for optimal combining of computed tomography and positron emission tomography imaging for attenuated correction and improved diagnosis.

Diagnostic imaging technologies allow images of internal features of a patient to be non-invasively obtained and may provide information about the function and integrity of the patient's internal structures. Diagnostic imaging systems may operate based on various physical principles, including the positron emission or transmission of radiation from the patient tissues. For example, single photon emission computed tomography (SPECT) and positron emission tomography (PET) may utilize a radiopharmaceutical that is administered to a patient and whose breakdown results in the positron emission of gamma rays from locations within the patient's body. The radiopharmaceutical is typically selected so as to be preferentially or differentially distributed in the body based on the physiological or biochemical processes in the body. For example, a radiopharmaceutical may be selected that is preferentially processed or taken up by tumor tissue. In such an example, the radiopharmaceutical will typically be disposed in greater concentrations around tumor tissue within the patient.

In the context of PET imaging, the radiopharmaceutical typically breaks down or decays within the patient, releasing a positron which annihilates when encountering an electron and produces a pair of gamma rays moving in opposite directions. In SPECT imaging, a single gamma ray is generated when the radiopharmaceutical breaks down or decays within the patient. These gamma rays interact with detection mechanisms within the respective PET or SPECT scanner, which allow the decay events to be localized, thereby providing a view of where the radiopharmaceutical is distributed throughout the patient. In this manner, a caregiver can visualize where in the patient the radiopharmaceutical is disproportionately distributed and may thereby identify where physiological structures and/or biochemical processes of diagnostic significance are located within the patient.

A PET imaging system generates images that represent the distribution of positron-emitting nuclides within the body of a patient. When a positron interacts with an electron by annihilation, the entire mass of the positron-electron pair is converted into two 511 keV photons. The photons are emitted in opposite directions along a line of response. The two annihilation photons (known as a coincidence pair) can be detected by detectors that are placed along the line of response on a detector ring. When these photons arrive and are detected at the detector elements at the same or nearly the same time, this is referred to as coincidence or coincidence event (COIN). An image is then generated, based on the acquired data that includes the annihilation photon detection information.

There are several types of motion that may result in suboptimal imaging and artifacts. Examples of these motion that may in suboptimal imaging and artifacts include patient motion, respiratory motion, cardiac contraction motion, and bowel motion. Of these, the respiratory motion, and the cardiac contraction motion are cyclic (or nearly cyclic) and can generally be gated or segmented to motion-free (or nearly motion-free) phases. This motion may result in misregistration between PET and CT data. PET and CT misregistration results in suboptimal imaging quality due to smeared lesions (e.g., lesion blurring and loss of contrast), attenuation correction artifacts (e.g., banana artifacts) due to misregistration between the PET and CT data, and diagnostic phase mismatch (e.g., organs in CT image do not correctly overlay on the PET emission data due to the time-averaging of the PET data versus the snapshot CT data).

PET/CT scans consist of CT and PET scans. The CT scan is rapid (e.g., approximately 5 to 20 seconds) while the duration of the PET scan spans several minutes. The CT scan is taken in a breath-hold mode (i.e., patient holds breath) to minimize motion blurring effect. However, the PET scans are too long to be scanned at a breath-hold mode. As such, an anatomical mismatch occurs between the PET and the CT scans may occur due to the respiration phase mismatch between PET and the CT data. This mismatch (e.g., registration mismatch) may result in sub-optimal attenuation correction (e.g., resulting in attenuation correction artifacts) and diagnostic challenges when the PET data and the CT data side by side.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for combining computed tomography and positron emission tomography imaging data is provided. The computer-implemented method includes reconstructing, via a processor, a computed tomography image of a region of interest of a subject based on computed tomography scan data acquired at a determined respiratory phase with a computed tomography scanner of a positron emission tomography-computed tomography imaging system. The computer-implemented method includes generating, via the processor, an attenuation map from the CT image. The computer-implemented method also includes obtaining, via the processor, positron emission tomography scan data of the region of interest of the subject acquired over multiple respiratory cycles with a positron emission tomography scanner of the positron emission tomography-computed tomography imaging system. The computer-implemented method further includes gating, via the processor, the positron emission tomography scan data into different respiratory phases. The computer-implemented method even further includes reconstructing, via the processor, a positron emission tomography image utilizing only the positron emission tomography scan data acquired at a same respiratory phase as the determined respiratory phase that the computed tomography image was acquired and the attenuation map from the CT image.

In another embodiment, a system for combining computed tomography and positron emission tomography imaging data is provided. The system includes a memory encoding processor-executable routines. The system also includes a processor configured to access the memory and to execute the processor-executable routines, wherein the processor-executable routines, when executed by the processor, cause the processor to perform actions. The actions include reconstructing a computed tomography image of a region of interest of a subject based on computed tomography scan data acquired at a determined respiratory phase with a computed tomography scanner of a positron emission tomography-computed tomography imaging system. The actions also include obtaining positron emission tomography scan data of the region of interest of the subject acquired over multiple respiratory cycles with a positron emission tomography scanner of the positron emission tomography-computed tomography imaging system. The actions further include gating the positron emission tomography scan data into different respiratory phases. The actions even further include reconstructing a positron emission tomography image utilizing only the positron emission tomography scan data acquired at a same respiratory phase as the determined respiratory phase that the computed tomography image was acquired. The actions still further include generating an attenuation map from the computed tomography image. The actions yet further include performing attenuation correction on the positron emission tomography image utilizing the attenuation map to generate an attenuation-corrected positron emission tomography image. The actions further include combining the computed tomography image with the attenuation-corrected positron emission tomography image to generate a motion artifact-free, motion-free combined image of the region of interest.

In a further embodiment, a non-transitory computer-readable medium is provided. The computer-readable medium includes processor-executable code that when executed by a processor, causes the processor to perform actions. The actions include reconstructing a computed tomography image of a region of interest of a subject based on computed tomography scan data acquired at a determined respiratory phase with a computed tomography scanner of a positron emission tomography-computed tomography imaging system. The actions also include obtaining positron emission tomography scan data of the region of interest of the subject acquired over multiple respiratory cycles with a positron emission tomography scanner of the positron emission tomography-computed tomography imaging system. The actions further include gating the positron emission tomography scan data into different respiratory phases. The actions even further include reconstructing a positron emission tomography image utilizing only the positron emission tomography scan data acquired at a same respiratory phase as the determined respiratory phase that the computed tomography image was acquired. The actions still further include generating an attenuation map from the computed tomography image. The actions yet further include performing attenuation correction on the positron emission tomography image utilizing the attenuation map to generate an attenuation-corrected positron emission tomography image. The actions further include combining the computed tomography image with the attenuation-corrected positron emission tomography image to generate a motion artifact-free, motion-free combined image of the region of interest.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a diagrammatical representation of an embodiment of a positron emission tomography imaging system, in accordance with aspects of the present disclosure;

FIG. 2 is a schematic diagram of an embodiment of a 3-D PET scanner, in accordance with aspects of the present disclosure;

FIGS. 3A, 3B, 3C, and 3D are schematic diagrams of perspective view, a trans-axial view, a side view, and another side view illustrating a ring difference, respectively, of a portion of a 3D PET scanner illustrating a line of response (LOR) in a PET imaging system, in accordance with aspects of the present disclosure;

FIG. 4 is a perspective view of a PET-computed tomography (CT) imaging system having the PET imaging system of FIG. 1, in accordance with aspects of the present disclosure;

FIG. 5 is a flowchart of a method for combining computed tomography and positron emission tomography imaging data, in accordance with aspects of the present disclosure;

FIG. 6 is a flowchart of a method for combining computed tomography and positron emission tomography imaging data (e.g., utilizing short positron emission tomography frames), in accordance with aspects of the present disclosure;

FIG. 7 is a flowchart of a method for combining computed tomography and positron emission tomography imaging data (e.g., utilizing a list mode file of the positron emission tomography scan data), in accordance with aspects of the present disclosure;

FIG. 8 is a flowchart of a method for combining computed tomography and positron emission tomography imaging data (e.g., utilizing a both a fast positron emission tomography scan and a long clinical tomography scan), in accordance with aspects of the present disclosure;

FIG. 9 is a schematic diagram illustrating the acquisition of data utilizing different types of positron emission tomography data acquisition relative to a respiratory cycle, in accordance with aspects of the present disclosure;

FIG. 10 is a graph comparing acquisition time and sensitivity for different types of positron emission tomography data acquisition, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Furthermore, the term processor or processing unit, as used herein, refers to any type of processing unit or system that can carry out the required calculations needed for the various embodiments, such as single or multi-core: CPU, Accelerated Processing Unit (APU), Graphics Board, DSP, FPGA, ASIC, cloud-based system, or a combination thereof, or a plurality of separate processing units. In addition, parts of the methods described below may be executed on different processors.

To solve the issue of misregistration between PET and CT data above, one may use the PET photons which are acquired at the exact respiratory phase that the CT data is acquired to generate the PET image. However, with conventional PET/CT scanner technology, the system sensitivity ranges from 10 to 50 counts per second (cps)/kilobecquerel (KBq), which are too few photons to match the CT phase. Therefore, the PET image will be too noisy. However, with large axial field of view (FOV) PET scanners, the sensitivity is increased ten times as compared to compared to conventional PET/CT scanners. Therefore, with the large axial field of view PET scanners, it is no longer a count starving modality. Thus, it may be feasible to use the PET photons to match the CT phase to generate high quality, combined images from PET/CT scans. In the context of the present disclosure, a large axial field of view PET scanner has an axial field of view of greater than 40 centimeters (cm). In certain embodiments, the axial field of view may be 64 centimeters. In certain embodiments, the axial field of view may be 120 centimeters.

In conventional PET, the PET scan duration dominates the time the PET/CT system is occupied by a patient, thus, limiting the system throughput. In contrast, with the large FOV PET scanner, patient setup and removal from the table dominates the time that the PET/CT system is occupied by a patient, thus, limiting system throughput. Thus, in large FOV PET scanners, increasing the PET scan duration can be tolerated in order to improve image quality without substantially increasing patient discomfort or substantially decreasing the system throughput.

The present disclosure provides systems and methods provide for optimal combining of computed tomography and positron emission tomography imaging data. In particular, the described systems and methods utilize positron emission tomography (e.g., PET) photons that are acquired at the exact respiratory phase that the CT data is acquired to generate a positron emission tomography image. The disclosed systems and methods include reconstructing a computed tomography image of a region of interest of a subject based on computed tomography scan data acquired at a determined respiratory phase with a computed tomography scanner of a positron emission tomography-computed tomography imaging system. In the context of the present disclosure, the term “motion free” refers only to the motion caused by cyclic motion (e.g., breathing, cardiac motion, both) that was gated. The disclosed systems and methods also include obtaining positron emission tomography scan data of the region of interest of the subject acquired over multiple respiratory cycles with a positron emission tomography scanner of the positron emission tomography-computed tomography imaging system. The disclosed systems and methods further include gating the positron emission tomography scan data into different respiratory phases. The disclosed systems and methods even further include reconstructing a positron emission tomography image utilizing only the positron emission tomography scan data acquired at a same respiratory phase as the determined respiratory phase that the computed tomography image was acquired. The disclosed systems and methods still further include generating an attenuation map from the computed tomography image. The disclosed systems and methods yet further include performing attenuation correction on the positron emission tomography image utilizing the attenuation map to generate an attenuation-corrected positron emission tomography image. The disclosed systems and methods further include combining the computed tomography image with the attenuation-corrected positron emission tomography image to generate a motion artifact-free, motion-free combined image of the region of interest. When the computed tomography scan is done during one phase of the respiratory cycle, and the PET data is limited to the same phase of the respiratory cycle, the images are free of cyclic motion due to respiratory motion (but may include cardiac cycle motion).

In certain embodiments, the disclosed systems and methods include, prior to reconstructing the positron emission tomography image, determining from the positron emission tomography scan data only the positron emission tomography scan data acquired at the same respiratory phase as the determined respiratory phase that the computed tomography image was acquired utilizing a list mode file of the positron emission tomography scan data. In certain embodiments, the disclosed systems and methods include, reconstructing a plurality of positron emission tomography frames from the positron emission tomography scan data, wherein each frame of the plurality of positron emission tomography frames spans less than 25 percent of a mean respiratory cycle. In certain embodiments, the disclosed systems and methods include, determining which positron emission tomography frames of the plurality of positron emission tomography frames were acquired at the same respiratory phase as the determined respiratory phase that the computed tomography image was acquired, wherein the positron emission tomography image is reconstructed from only the positron emission tomography frames of the plurality of positron emission tomography frames that were acquired at the same respiratory phase as the determined respiratory phase that the computed tomography image was acquired.

In certain embodiments, the disclosed systems and methods include, prior to obtaining the positron emission tomography scan data, obtaining initial positron emission tomography scan data acquired with the positron emission tomography scanner simultaneously with the acquisition of the computed tomography scan data acquired at the determined respiratory phase with the computed tomography scanner so that the initial positron emission tomography scan data and the computed tomography scan data are acquired at a same respiratory phase. In certain embodiments, the disclosed systems and method include, prior to reconstructing the positron emission tomography image, determining from the positron emission tomography scan data only the positron emission tomography scan data from a list mode file of the positron emission tomography scan data that matches the initial positron emission tomography scan data. In certain embodiments, the disclosed systems and methods include, prior to reconstructing the positron emission tomography image, reconstructing a plurality of positron emission tomography frames from the positron emission tomography scan data, wherein each frame of the plurality of positron emission tomography frames spans less than 25 percent of a mean respiratory cycle. In certain embodiments, the disclosed systems and methods include, determining which positron emission tomography frames of the plurality of positron emission tomography frames that match the initial positron emission tomography scan data, wherein the positron emission tomography image is reconstructed from only the positron emission tomography frames of the plurality of positron emission tomography frames that match the initial positron emission tomography scan data.

It should be noted that that although the disclosed embodiments are discussed in the context of matching respiratory phase, the disclosed embodiments can be extended to matching between cardiac phase for acquired positron emission tomography and computed tomography data. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired. When the computed tomography scan is done during one phase of the cardiac cycle, and the PET data is limited to the same phase of the cardiac cycle, the images are free of cyclic motion due to cardiac motion.

The disclosed embodiments optimize the combining of PET and CT imaging data (e.g., due to matching data acquired at a same respiration phase). The disclosed embodiments improve the image quality derived from the PET data due to improved attenuation correction. The disclosed embodiments also improve diagnosis due to the full optimized combining of the PET and CT imaging data.

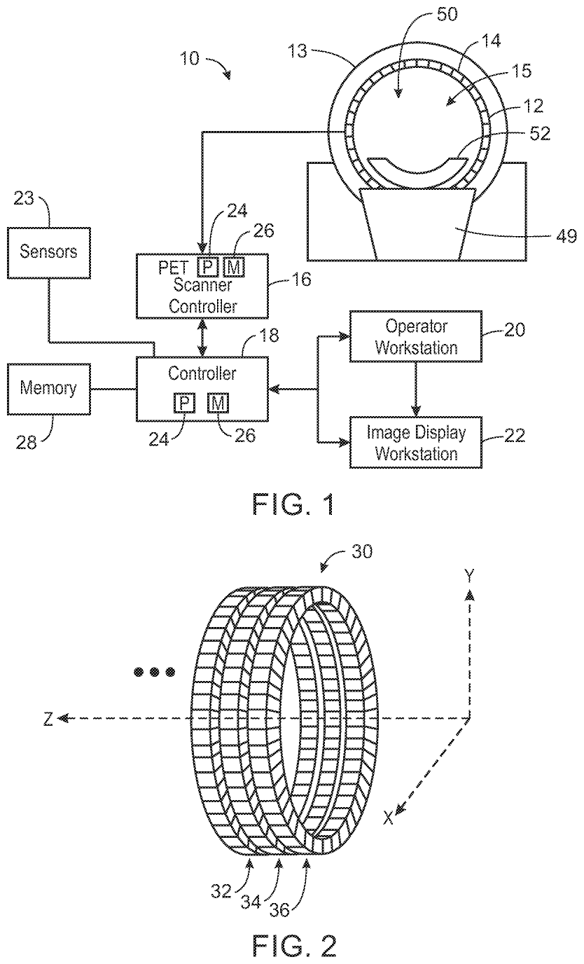

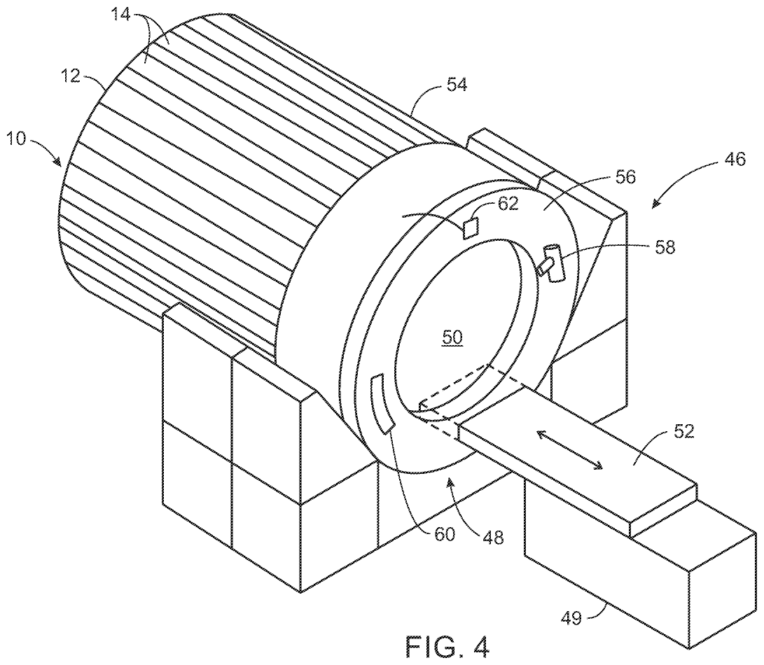

With the foregoing in mind and turning now to the drawings, FIG. 1 depicts a PET imaging system 10 operating in accordance with certain aspects of the present disclosure. The PET imaging system of FIG. 1 may be utilized with a dual-modality imaging system such as a PET-CT imaging system described in FIG. 4.

Returning now to FIG. 1, the depicted PET imaging system 10 includes a detector array 12. The detector array 12 of the PET imaging system 10 typically includes a number of detector modules or detector assemblies (generally designated by reference numeral 14) arranged in a plurality of rings as depicted in FIG. 1. Each detector module 14 may include a scintillator block (e.g., having a plurality of scintillation crystals) and a photomultiplier tube (PMT) or other light sensor or photosensor (e.g. silicon avalanche photodiode, solid state photomultiplier, etc.). In certain embodiments, a respective photosensor is associated with a respective scintillator crystal. In some embodiments, direct conversion, solid-state photon detectors can be used. The PET imaging system 10 includes a gantry 13 that is configured to support a full ring annular detector array 12 thereon. The detector array 12 is positioned around the central opening/bore 50 and can be controlled to perform a normal “emission scan” in which positron annihilation events are counted. To this end, the detector modules 14 forming the detector array 12 generally generate intensity output signals corresponding to each annihilation photon (which are acquired by acquisition circuitry coupled to the detector modules 14).

The depicted PET system 10 also includes a PET scanner controller 16, a controller 18, an operator workstation 20, and an image display workstation 22 (e.g., for displaying an image). In certain embodiments, the PET scanner controller 16, controller 18, operator workstation 20, and image display workstation 22 may be combined into a single unit or device or fewer units or devices. The PET system 10 also includes a table 52 coupled to a table base 49. The table 52 is configured to be moved into and out of the opening/bore 50 with the patient on the table 52. In certain embodiments, one or more sensors 23 may be coupled to the controller to provide signals to enable gating (e.g., respiratory gating or cardiac gating) of acquired positron emission tomography data. For example, the sensors 23 may include breathing sensors or one or more straps disposed about a chest or abdomen of the subject. In certain embodiments, an electrocardiogram (ECG) acquisition unit may be coupled to controller 18 to provide ECG signals acquired from a subject (e.g., during a PET scan) for utilization in the techniques described below.

The PET scanner controller 16, which is coupled to the detector 12, may be coupled to the controller 18 to enable the controller 18 to control operation of the PET scanner controller 16. Alternatively, the PET scanner controller 16 may be coupled to the operator workstation 20 which controls the operation of the PET scanner controller 16. In operation, the controller 18 and/or the workstation 20 controls the real-time operation of the PET system 10. In certain embodiments the controller 18 and/or the workstation 20 may control the real-time operation of another imaging modality (e.g., the CT imaging system 56 in FIG. 4) to enable the simultaneous and/or separate acquisition of image data from the different imaging modalities. One or more of the PET scanner controller 16, the controller 18, and/or the operation workstation 20 may include a processor 24 and/or memory 26. In certain embodiments, the PET system 10 may include a separate memory 28. The detector 12, PET scanner controller 16, the controller 18, and/or the operation workstation 20 may include detector acquisition circuitry for acquiring image data from the detector 12 and image reconstruction and processing circuitry for image processing. The circuitry may include specially programmed hardware, memory, and/or processors.

The processor 24 may include multiple microprocessors, one or more “general-purpose” microprocessors, one or more special-purpose microprocessors, and/or one or more application specific integrated circuits (ASICS), system-on-chip (SoC) device, or some other processor configuration. For example, the processor 24 may include one or more reduced instruction set (RISC) processors or complex instruction set (CISC) processors. The processor 24 may execute instructions to carry out the operation of the PET system 10. These instructions may be encoded in programs or code stored in a tangible non-transitory computer-readable medium (e.g., an optical disc, solid state device, chip, firmware, etc.) such as the memory 26, 28. In certain embodiments, the memory 26 may be wholly or partially removable from the controller 16, 18.

As described in greater detail below, the processor 24 is configured for optimal combining of computed tomography and positron emission tomography imaging data. In particular, the processor 24 is configured to utilize positron emission tomography (e.g., PET) photons that are acquired at the exact respiratory phase that the CT data is acquired to generate a positron emission tomography image. The processor 24 is configured to reconstruct a computed tomography image of a region of interest of a subject based on computed tomography scan data acquired at a determined respiratory phase with a computed tomography scanner of a positron emission tomography-computed tomography imaging system. The processor 24 is also configured to obtain positron emission tomography scan data of the region of interest of the subject acquired over multiple respiratory cycles with a positron emission tomography scanner of the positron emission tomography-computed tomography imaging system. The processor 24 is further configured to gate the positron emission tomography scan data into different respiratory phases. The processor 24 is even further configured to reconstruct a positron emission tomography image utilizing only the positron emission tomography scan data acquired at a same respiratory phase as the determined respiratory phase that the computed tomography image was acquired. The processor 24 is still further configured to generate an attenuation map from the computed tomography image. The processor 24 is yet further configured to perform attenuation correction on the positron emission tomography image utilizing the attenuation map to generate an attenuation-corrected positron emission tomography image. The processor 24 is further configured to combine the computed tomography image with the attenuation-corrected positron emission tomography image to generate a motion artifact-free, motion-free combined image of the region of interest.

In certain embodiments, the processor 24 is configured, prior to reconstructing the positron emission tomography image, to determine from the positron emission tomography scan data only the positron emission tomography scan data acquired at the same respiratory phase as the determined respiratory phase that the computed tomography image was acquired utilizing a list mode file of the positron emission tomography scan data. In certain embodiments, the processor 24 is configured, to reconstruct a plurality of positron emission tomography frames from the positron emission tomography scan data, wherein each frame of the plurality of positron emission tomography frames spans less than 25 percent of a mean respiratory cycle. In certain embodiments, the processor 24 is configured, prior to reconstructing the positron emission tomography image, to determine which positron emission tomography frames of the plurality of positron emission tomography frames were acquired at the same respiratory phase as the determined respiratory phase that the computed tomography image was acquired, wherein the positron emission tomography image is reconstructed from only the positron emission tomography frames of the plurality of positron emission tomography frames that were acquired at the same respiratory phase as the determined respiratory phase that the computed tomography image was acquired.

In certain embodiments, the processor 24 is configured, prior to obtaining the positron emission tomography scan data, to obtain initial positron emission tomography scan data acquired with the positron emission tomography scanner simultaneously with the acquisition of the computed tomography scan data acquired at the determined respiratory phase with the computed tomography scanner so that the initial positron emission tomography scan data and the computed tomography scan data are acquired at a same respiratory phase. In certain embodiments, the processor 24 is configured, prior to reconstructing the positron emission tomography image, to determine from the positron emission tomography scan data only the positron emission tomography scan data from a list mode file of the positron emission tomography scan data that matches the initial positron emission tomography scan data. In certain embodiments, the processor 24 is configured, prior to reconstructing the positron emission tomography image, to reconstruct a plurality of positron emission tomography frames from the positron emission tomography scan data, wherein each frame of the plurality of positron emission tomography frames spans less than 25 percent of a mean respiratory cycle. In certain embodiments, the processor 24 is configured, to determine which positron emission tomography frames of the plurality of positron emission tomography frames that match the initial positron emission tomography scan data, wherein the positron emission tomography image is reconstructed from only the positron emission tomography frames of the plurality of positron emission tomography frames that match the initial positron emission tomography scan data.

In certain embodiments, the processor 24 is configured to perform matching between acquired positron emission tomography and computed tomography data for a particular cardiac phase. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired.

By way of example, PET imaging is primarily used to measure metabolic activities that occur in tissues and organs and, in particular, to localize aberrant metabolic activity. In PET imaging, the patient is typically injected with a solution that contains a radioactive tracer. The solution is distributed and absorbed throughout the body in different degrees, depending on the tracer employed and the functioning of the organs and tissues. For instance, tumors typically process more glucose than a healthy tissue of the same type. Therefore, a glucose solution containing a radioactive tracer may be disproportionately metabolized by a tumor, allowing the tumor to be located and visualized by the radioactive emissions. In particular, the radioactive tracer emits positrons that interact with and annihilate complementary electrons to generate pairs of gamma rays. In each annihilation reaction, two gamma rays traveling in opposite directions are emitted. In a PET imaging system 10, the pair of gamma rays are detected by the detector array 12 configured to ascertain that two gamma rays detected sufficiently close in time are generated by the same annihilation reaction. Due to the nature of the annihilation reaction, the detection of such a pair of gamma rays may be used to determine the line of response along which the gamma rays traveled before impacting the detector, allowing localization of the annihilation event to that line. By detecting a number of such gamma ray pairs, and calculating the corresponding lines traveled by these pairs, the concentration of the radioactive tracer in different parts of the body may be estimated and a tumor, thereby, may be detected. Therefore, accurate detection and localization of the gamma rays forms a fundamental and foremost objective of the PET imaging system 10.

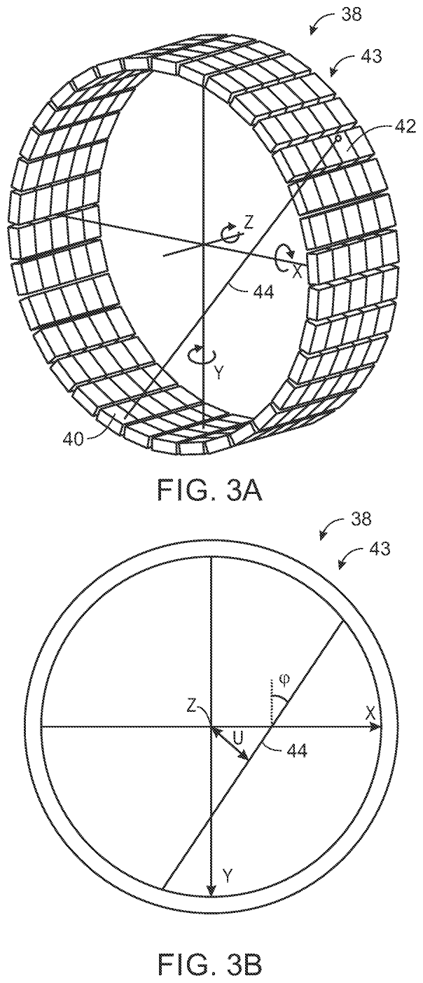

Data associated with coincidence events along a number of LORs may be collected and further processed to reconstruct three-dimensional (3-D) tomographic images. Modern PET scanners, specifically large AFOV scanners, operate in a 3-D PET mode, where coincidence events from different detector rings positioned along the axial direction are counted to obtain tomographic images. For example, a PET scanner 30 with multiple detector rings is shown in FIG. 2, where the individual detectors and photosensors are not shown. The PET scanner detector 30 includes a plurality of detector rings. In FIG. 2 only three detector rings 32, 34 and 36 of the plurality of detector rings are shown. The number of detector rings may vary (e.g., 2, 3, 4, 5, or more detector rings. In a larger AFOV PET detector, the number of detector rings is greater than 10 rings. Most narrow AFOV PET cameras have a sensitivity along their AFOV having the shape of a triangle, while are large AFOV PET scanner (e.g., having greater than 10 detector rings) can have a sensitivity along the AFOV having the shape of a trapezoid. However, some large AFOV PET scanner are having sensitivity along their AFOV having the shape of a triangle. In the disclosed embodiments, coincidence events may occur in different detector rings of different gantry segments of the modular gantry along the axial direction.

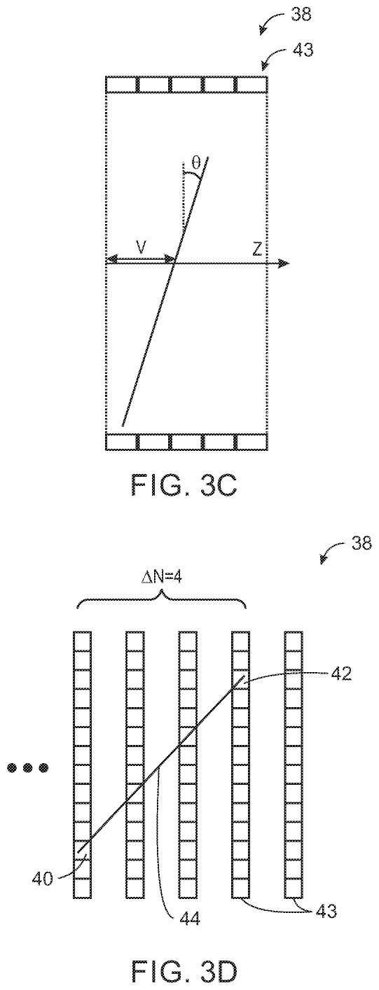

Traditionally, data associated with coincidence events are stored in the form of sinograms based on their corresponding LORs. For example, in a 3-D PET scanner 38 like the one illustrated in FIGS. 3A-D, if a pair of coincidence events are detected by detectors 40 and 42 in different detector rings 43, an LOR may be established as a straight line 44 linking the two detectors 40, 42. It should be noted for simplicity only five rings 53 are shown and only two of the five rings 53 are marked for simplicity. In a 3-D PET scanner, an LOR is defined by four coordinates (u, φ, v, θ), wherein the first coordinate u is the radial distance of the LOR from the center axis of the detector, the second coordinate φ is the trans-axial angle between the LOR and the X-axis, the third coordinate v is the distance of the LOR from the center of the detector rings along the Z-axis, and the fourth coordinate θ is the axial angle between the LOR and the center axis (or Z-axis) of the detector rings. As the PET scanner continues to detect coincidence events along various LORs, these events may be binned and accumulated in their corresponding elements. In this case, the detected coincidence events are stored in a 4-D sinogram (u, φ, v, θ), where each element of which holds an event count for a specific LOR.

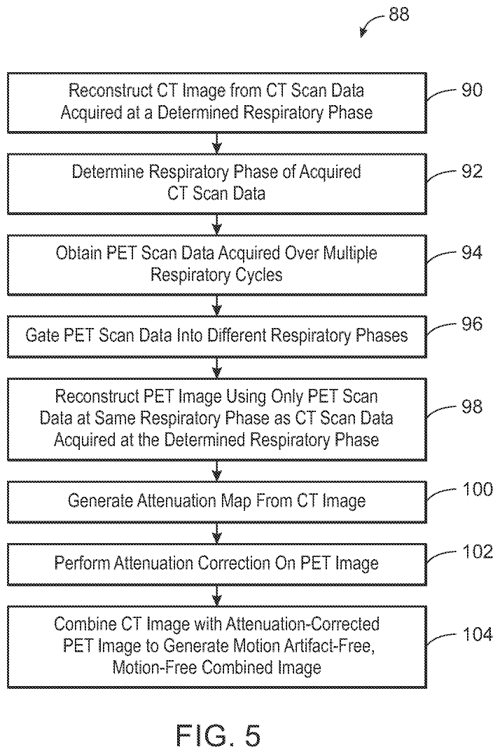

As illustrated in FIGS. 3C and 3D (which are a side views of a 3-D PET scanner 38 having a plurality of detector rings 43, (only five rings are drawn, and only two of the five are marked to avoid cluttering the drawing), a pair of coincidence events are detected by two detectors 40 and 42 on different detector rings 43, an LOR may be established as a straight line 44 linking the two detectors 40 and 42. As depicted in the example in FIG. 3D, there is a ring difference (ΔN) of 4. ΔN-max (which is implemented in software) defines the maximum number of adjacent detector rings 53 that are taken into account in generating an image. Coincidence events detected with ΔN>ΔN-max are not considered when reconstructing the 3D image. This maximum ring difference, ΔN-max, defines the size of edge regions 80 (i.e., number of rings to utilize for each edge region 80) of a sensitivity profile 76 discussed in FIGS. 5A-D. Optionally, ΔN-max is a user selected parameter, or is associated with a specific diagnostic protocol, or selected based on patient parameters such as weight, height, body mass index (BMI), etc. When ΔN-max is selected to be equal to the number of rings, the sensitivity profile is triangular.

As mentioned above, the PET imaging system 10 may be incorporated into a dual-modality imaging system such as the PET-CT imaging system 46 in FIG. 4. Referring now to FIG. 4, the PET-CT imaging system 46 includes the PET system 10 and a CT system 48 positioned in fixed relationship to one another. The PET system 10 and CT system 48 are aligned to allow for translation of a patient (not shown) therethrough. In use, a patient is positioned within a bore 50 of the PET-CT imaging system 46 (via a table 52 controlled by a table controller that is controlled by the controller 18 and/or operator workstation 20 described in FIG. 2) to image a region of interest of the patient as is known in the art.

The PET imaging system 10 includes a gantry 54 that is configured to support a full ring annular detector array 12 thereon (e.g., including the plurality of detector assemblies 14 in FIG. 1). The detector array 12 is positioned around the central opening/bore 50 and can be controlled to perform a normal “emission scan” in which positron annihilation events are counted. To this end, the detectors 14 forming array 12 generally generate intensity output signals corresponding to each detected annihilation photon.

The CT system 48 includes a rotatable gantry 56 having an X-ray source 58 thereon that projects a beam of X-rays toward a detector assembly 60 on the opposite side of the gantry 56. The detector assembly 60 senses the projected X-rays that pass through a patient and measures the intensity of an impinging X-ray beam and hence the attenuated beam as it passes through the patient. During a scan to acquire X-ray projection data, rotatable gantry 56 and the components mounted thereon rotate about a center of rotation. In certain embodiments, the CT system 48 may be controlled by the controller 18 and/or operator workstation 20 described in FIG. 2. In certain embodiments, the PET system 10 and the CT system 48 may share a single gantry. Image data may be acquired simultaneously and/or separately with the PET system 10 and the CT system 48. In certain embodiments, the CT system 48 may include an optical imaging system 62 (e.g., camera) coupled to a stationary portion of the CT system 48 (e.g., outer cover of CT system 48) for optically imaging the patient to assist in setting up the patient for a scan.

FIG. 5 is a flowchart of a method 88 for combining computed tomography and positron emission tomography imaging data. One or more steps of the method 88 may be performed by processing circuitry of the imaging systems discussed above. One or more of the steps of the method 88 may be performed simultaneously or in a different order from the order depicted in FIG. 5. In certain embodiments, the method 88 may be performed utilizing a dual-modality imaging system such as a positron emission tomography/CT imaging system (e.g., PET-CT imaging system 46 in FIG. 4 or a SPECT CT imaging system).

The method 88 includes reconstructing a computed tomography image of a region of interest of a subject (e.g., patient) based on computed tomography scan data acquired at a determined respiratory phase (e.g., inspiratory phase, inspiratory pause, expiratory phase, expiratory pause) with a computed tomography scanner of a positron emission tomography-computed tomography imaging system (block 90). In certain embodiments, the computed tomography scan data is acquired under a breath-hold mode (e.g., subject holding breath) to minimize the motion blurring effect. In certain embodiments, the computed tomography acquisition is short enough to acquire the computed tomography scan data for one phase of the respiratory cycle. In certain embodiments, the computed tomography acquisition is conducted utilizing fast rotating computed tomography as known in the art. In certain embodiments, the method 88 includes determining the respiratory phase of the acquired computed tomography data (block 92). In certain embodiments, one or more sensors may be utilized to determine the respiratory phase when the computed tomography data was acquired. For example, in certain embodiments, determining the respiratory phase may be conducted by analyzing heart rate variations. In certain embodiments, determining the respiratory phase may be data-driven, that is, extracted from the PET emission data.

The method 88 also includes obtaining positron emission tomography scan data of the region of interest of the subject acquired over multiple respiratory cycles with a positron emission tomography scanner of the positron emission tomography-computed tomography imaging system (block 94). For example, the positron emission tomography scan (e.g., long clinical scan) may have a duration of approximately 90 seconds. Acquiring the positron emission tomography scan data over multiple respiratory cycles and over this scan duration enables a portion of the positron emission tomography data to be discarded (if needed) yet provide enough data for sufficient positron emission tomography imaging quality. The method 88 further includes gating (e.g., respiratory gating by phase or amplitude) the positron emission tomography scan data into different respiratory phases (block 96). In certain embodiments, respiratory gating may be achieved from short time reconstruction of data. In certain embodiments, respiratory gating may be derived from cardiac rate variation in acquired electrocardiogram data. In certain embodiments, respiratory gating may be performed utilizing data from sensors (e.g., breathing sensors or one or more straps disposed about a chest or abdomen of the subject).

The method 88 even further includes reconstructing a positron emission tomography image utilizing only the positron emission tomography scan data acquired at a same respiratory phase as the determined respiratory phase that the computed tomography image was acquired in block 90 (block 98). The method 88 still further includes generating an attenuation map from the computed tomography image (block 100). The method 88 yet further includes performing attenuation correction on the positron emission tomography image utilizing the attenuation map to generate an attenuation-corrected positron emission tomography image (block 102). The method 88 further includes combining the computed tomography image with the attenuation-corrected positron emission tomography image to generate a motion artifact-free, motion-free combined image (e.g., diagnostic) of the region of interest (block 104). In particular, the computed tomography image is overlaid on the attenuation corrected positron emission tomography image to generate the motion artifact-free, motion-free combined image.

In certain embodiments, the method 88 may be modified to be utilized with cardiac phase matching instead of respiration phase matching. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired.

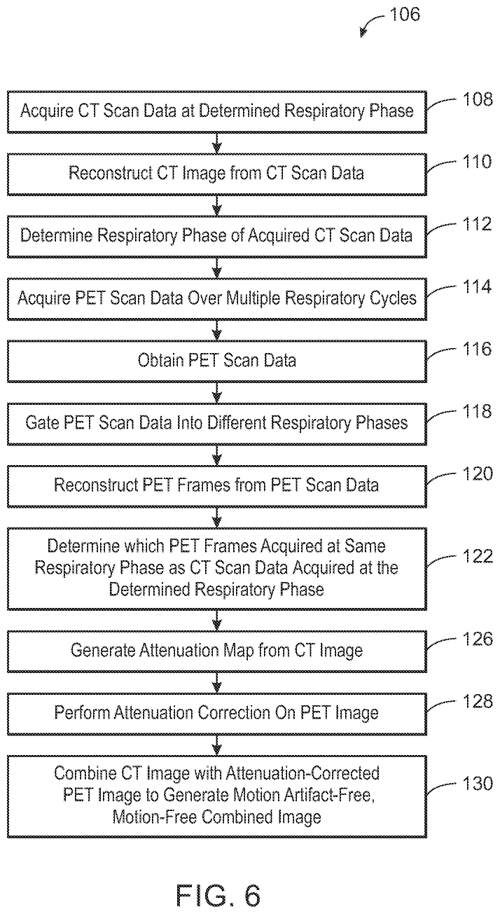

FIG. 6 is a flowchart of a method 106 for combining computed tomography and positron emission tomography imaging data (e.g., utilizing short positron emission tomography frames). One or more steps of the method 106 may be performed by processing circuitry of the imaging systems discussed above. One or more of the steps of the method 106 may be performed simultaneously or in a different order from the order depicted in FIG. 6. In certain embodiments, the method 106 may be performed utilizing a dual-modality imaging system such as a positron emission tomography/CT imaging system (e.g., PET-CT imaging system 46 in FIG. 4 or a SPECT CT imaging system).

The method 106 includes acquiring/obtaining computed tomography scan data of a region of interest of a subject (e.g., patient) at a determined respiratory phase (e.g., inspiratory phase, inspiratory pause, expiratory phase, expiratory pause) with a computed tomography scanner of a positron emission tomography-computed tomography imaging system (block 108). In certain embodiments, the computed tomography scan data is acquired under a breath-hold mode (e.g., subject holding breath) to minimize the motion blurring effect. In certain embodiments, the computed tomography acquisition is short enough to acquire the computed tomography scan data for one phase of the respiratory cycle. In certain embodiments, the computed tomography acquisition is conducted utilizing fast rotating computed tomography as known in the art. The method 106 also includes reconstructing a computed tomography image of the region of interest of the subject based on the computed tomography scan data acquired at the determined respiratory phase (block 110). In certain embodiments, the method 106 includes determining the respiratory phase of the acquired computed tomography data (block 112). In certain embodiments, one or more sensors may be utilized to determine the respiratory phase when the computed tomography data was acquired.

The method 106 includes acquiring positron emission tomography scan data of the region of interest of the subject over multiple respiratory cycles with a positron emission tomography scanner of the positron emission tomography-computed tomography imaging system (block 114). For example, the positron emission tomography scan (e.g., long clinical scan) may have a duration of approximately 90 seconds. Acquiring the positron emission tomography scan data over multiple respiratory cycles and over this scan duration enables a portion of the positron emission tomography data to be discarded (if needed) yet provide enough data for sufficient positron emission tomography imaging quality. The method 106 also includes obtaining the positron emission tomography scan data of the region of interest of the subject acquired over the multiple respiratory cycles (block 116). The method 106 further includes gating (e.g., respiratory gating by phase or amplitude) the positron emission tomography scan data into different respiratory phases (block 118). In certain embodiments, respiratory gating may be achieved from short time reconstruction of data. In certain embodiments, respiratory gating may be derived from cardiac rate variation in acquired electrocardiogram data. In certain embodiments, respiratory gating may be performed utilizing data from sensors (e.g., breathing sensors or one or more straps disposed about a chest or abdomen of the subject).

The method 106 includes reconstructing a plurality of positron emission tomography frames from the positron emission tomography scan data, wherein each frame of the plurality of positron emission tomography frames spans less than 25 percent of a mean respiratory cycle (block 120). In certain embodiments, each frame spans approximately a second or less. In certain embodiments, each frame spans approximately 0.5 second or less. The method 106 also includes determining which positron emission tomography frames of the plurality of positron emission tomography frames were acquired at the same respiratory phase as the determined respiratory phase that the computed tomography image was acquired at block 108 to utilize as the positron emission tomography image (block 122). The motion-free emission tomography image is reconstructed from only the positron emission tomography frames of the plurality of emission tomography frames (which have been grouped via summation) that were acquired at the same respiratory phase as the determined respiratory phase that the motion-free computed tomography image was acquired.

The method 106 still further includes generating an attenuation map from the computed tomography image (block 126). The method 106 yet further includes performing attenuation correction on the positron emission tomography image utilizing the attenuation map to generate an attenuation-corrected positron emission tomography image (block 128). The method 106 further includes combining the computed tomography image with the attenuation-corrected positron emission tomography image to generate a motion artifact-free, motion-free combined image (e.g., diagnostic) of the region of interest (block 130). In particular, the computed tomography image is overlaid on the attenuation corrected positron emission tomography image to generate the motion artifact-free, motion-free combined image.

In certain embodiments, the method 106 may be modified to be utilized with cardiac phase matching instead of respiration phase matching. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired.

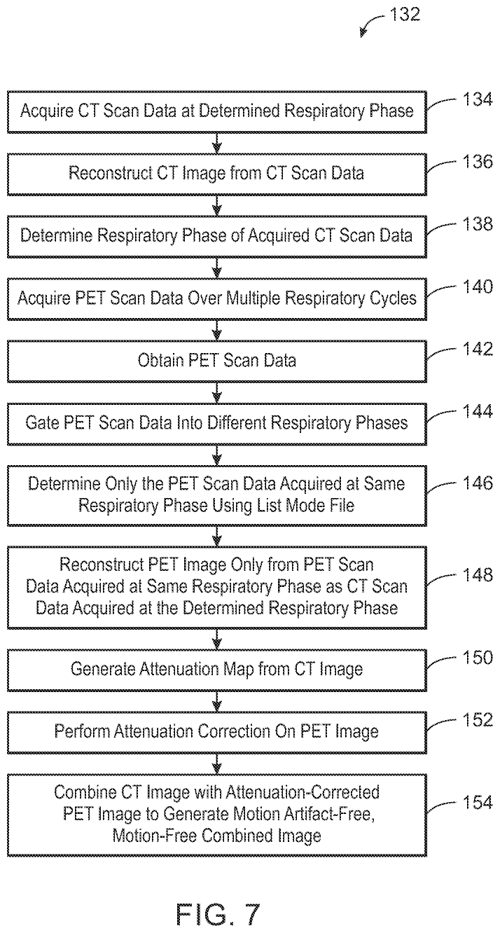

FIG. 7 is a flowchart of a method 132 for combining computed tomography and positron emission tomography imaging data (e.g., utilizing a list mode file of the positron emission tomography scan data). One or more steps of the method 132 may be performed by processing circuitry of the imaging systems discussed above. One or more of the steps of the method 132 may be performed simultaneously or in a different order from the order depicted in FIG. 7. In certain embodiments, the method 132 may be performed utilizing a dual-modality imaging system such as a positron emission tomography/CT imaging system (e.g., PET-CT imaging system 46 in FIG. 4 or a SPECT CT imaging system).

The method 132 includes acquiring/obtaining computed tomography scan data of a region of interest of a subject (e.g., patient) at a determined respiratory phase (e.g., inspiratory phase, inspiratory pause, expiratory phase, expiratory pause) with a computed tomography scanner of a positron emission tomography-computed tomography imaging system (block 134). In certain embodiments, the computed tomography scan data is acquired under a breath-hold mode (e.g., subject holding breath) to minimize the motion blurring effect. In certain embodiments, the computed tomography acquisition is short enough to acquire the computed tomography scan data for one phase of the respiratory cycle. In certain embodiments, the computed tomography acquisition is conducted utilizing fast rotating computed tomography as known in the art. The method 132 also includes reconstructing a computed tomography image of the region of interest of the subject based on the computed tomography scan data acquired at the determined respiratory phase (block 136). In certain embodiments, the method 132 includes determining the respiratory phase of the acquired computed tomography data (block 138). In certain embodiments, one or more sensors may be utilized to determine the respiratory phase when the computed tomography data was acquired.

The method 132 includes acquiring positron emission tomography scan data of the region of interest of the subject over multiple respiratory cycles with a positron emission tomography scanner of the positron emission tomography-computed tomography imaging system (block 140). For example, the positron emission tomography scan (e.g., long clinical scan) may have a duration of approximately 90 seconds. Acquiring the positron emission tomography scan data over multiple respiratory cycles and over this scan duration enables a portion of the positron emission tomography data to be discarded (if needed) yet provide enough data for sufficient positron emission tomography imaging quality. The method 132 also includes obtaining the positron emission tomography scan data of the region of interest of the subject acquired over the multiple respiratory cycles (block 142). The method 132 further includes gating (e.g., respiratory gating by phase or amplitude) the positron emission tomography scan data into different respiratory phases (block 144). In certain embodiments, respiratory gating may be achieved from short time reconstruction of data. In certain embodiments, respiratory gating may be derived from cardiac rate variation in acquired electrocardiogram data. In certain embodiments, respiratory gating may be performed utilizing data from sensors (e.g., breathing sensors or one or more straps disposed about a chest or abdomen of the subject).

The method 132 includes determining from the positron emission tomography scan data only the positron emission tomography scan data acquired at the same respiratory phase as the determined respiratory phase that the computed tomography image was acquired at block 134 utilizing a list mode file of the positron emission tomography scan data. (block 146). The method 132 even further includes reconstructing a positron emission tomography image utilizing only the positron emission tomography scan data acquired at a same respiratory phase as the determined respiratory phase that the computed tomography image was acquired (block 148).

The method 132 still further includes generating an attenuation map from the computed tomography image (block 150). The method 132 yet further includes performing attenuation correction on the positron emission tomography image utilizing the attenuation map to generate an attenuation-corrected positron emission tomography image (block 152). The method 132 further includes combining the computed tomography image with the attenuation-corrected positron emission tomography image to generate a motion artifact-free, motion-free combined image (e.g., diagnostic) of the region of interest (block 154). In particular, the computed tomography image is overlaid on the attenuation corrected positron emission tomography image to generate the motion artifact-free, motion-free combined image.

In certain embodiments, the method 132 may be modified to be utilized with cardiac phase matching instead of respiration phase matching. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired.

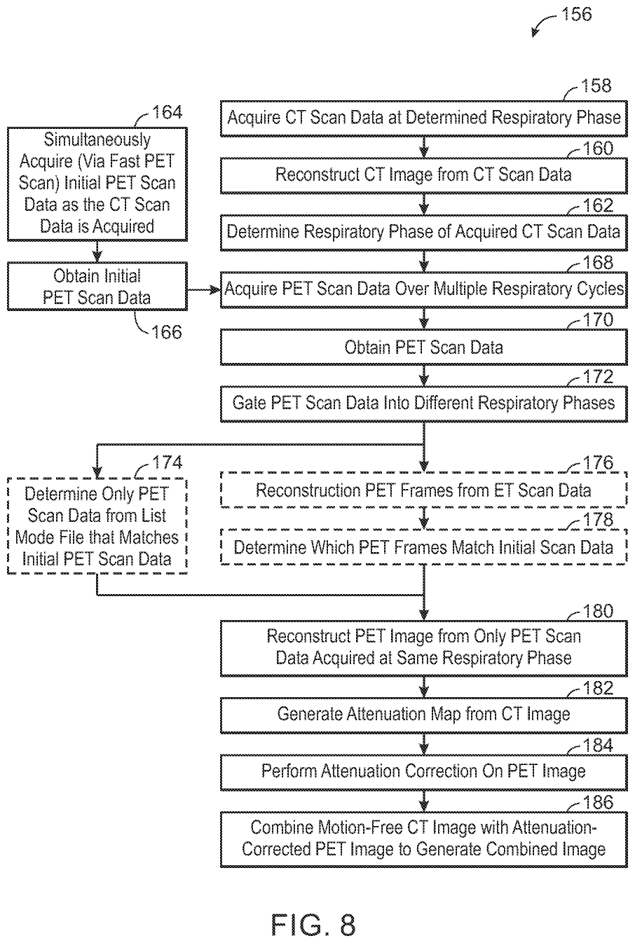

FIG. 8 is a flowchart of a method 156 for combining computed tomography and positron emission tomography imaging data (e.g., utilizing a both a fast positron emission tomography scan and a long clinical tomography scan). One or more steps of the method 156 may be performed by processing circuitry of the imaging systems discussed above. One or more of the steps of the method 156 may be performed simultaneously or in a different order from the order depicted in FIG. 8. In certain embodiments, the method 156 may be performed utilizing a dual-modality imaging system such as a positron emission tomography/CT imaging system (e.g., PET-CT imaging system 46 in FIG. 4 or a SPECT CT imaging system).

The method 156 includes acquiring/obtaining computed tomography scan data of a region of interest of a subject (e.g., patient) at a determined respiratory phase (e.g., inspiratory phase, inspiratory pause, expiratory phase, expiratory pause) with a computed tomography scanner of a positron emission tomography-computed tomography imaging system (block 158). In certain embodiments, the computed tomography scan data is acquired under a breath-hold mode (e.g., subject holding breath) to minimize the motion blurring effect. In certain embodiments, the computed tomography acquisition is short enough to acquire the computed tomography scan data for one phase of the respiratory cycle. In certain embodiments, the computed tomography acquisition is conducted utilizing fast rotating computed tomography as known in the art. The method 156 also includes reconstructing a computed tomography image of the region of interest of the subject based on the computed tomography scan data acquired at the determined respiratory phase (block 160). In certain embodiments, the method 156 includes determining the respiratory phase of the acquired computed tomography data (block 162). In certain embodiments, one or more sensors may be utilized to determine the respiratory phase when the computed tomography data was acquired.

The method 156 also includes simultaneously acquiring initial positron emission tomography scan data (e.g., via a fast positron emission tomography scan) with the positron emission scanner as the computed tomography scan data is being acquired at the determined respiratory phase with the computed tomography scanner (block 164). The positron emission tomography scan is the same duration as the computed tomography scan. Thus, the initial positron emission tomography scan data is acquired for the same determined respiratory phase. The method 156 includes obtaining the initial positron emission tomography scan data (block 166). As noted below, the initial positron emission tomography data enables identification of matching positron emission tomography scan data for the determined respiratory phase.

The method 156 includes acquiring positron emission tomography scan data of the region of interest of the subject over multiple respiratory cycles with a positron emission tomography scanner of the positron emission tomography-computed tomography imaging system (block 168). For example, the positron emission tomography scan (e.g., long clinical scan) may have a duration of approximately 90 seconds. Acquiring the positron emission tomography scan data over multiple respiratory cycles and over this scan duration enables a portion of the positron emission tomography data to be discarded (if needed) yet provide enough data for sufficient positron emission tomography imaging quality. The method 156 also includes obtaining the positron emission tomography scan data of the region of interest of the subject acquired over the multiple respiratory cycles (block 170). The method 156 further includes gating (e.g., respiratory gating by phase or amplitude) the positron emission tomography scan data into different respiratory phases (block 172). In certain embodiments, respiratory gating may be achieved from short time reconstruction of data. In certain embodiments, respiratory gating may be derived from cardiac rate variation in acquired electrocardiogram data. In certain embodiments, respiratory gating may be performed utilizing data from sensors (e.g., breathing sensors or one or more straps disposed about a chest or abdomen of the subject).

In certain embodiments, the method 156 further includes determining from the positron emission tomography scan data only the positron emission tomography scan data from a list mode file of the positron emission tomography scan data that matches the initial positron emission tomography scan data (block 174). In certain embodiments the method 156 further includes reconstructing a plurality of positron emission tomography frames from the positron emission tomography scan data, wherein each frame of the plurality of positron emission tomography frames spans less than 25 percent of a mean respiratory cycle (block 176). In certain embodiments, each frame spans approximately a second or less. In certain embodiments, each frame spans approximately 0.5 second or less. In certain embodiments, a plurality of positron emission tomography frames may be reconstructed from the initial positron emission tomography data. In certain embodiments, the method 156 further includes determining which positron emission tomography frames of the plurality of positron emission tomography frames that match the initial positron emission tomography scan data (e.g., positron emission tomography frames of the initial positron emission tomography data of the determined respiratory phase) (block 178).

The method 156 even further includes reconstructing a positron emission tomography image utilizing only the positron emission tomography scan data acquired at a same respiratory phase as the determined respiratory phase that the computed tomography image was acquired (and that matches the phase of the initial positron emission tomography data) (block 180). In embodiments, where positron emission frames are utilized, the positron emission tomography image is reconstructed when the frames were reconstructed (and the determined frames combined via summation). In certain embodiments, when the positron emission tomography frames of the positron emission tomography are utilized, the positron emission tomography image is reconstructed from only the positron emission tomography frames of the plurality of positron emission tomography frames that match the initial positron emission tomography scan data.

The method 156 still further includes generating an attenuation map from the computed tomography image (block 182). The method 156 yet further includes performing attenuation correction on the positron emission tomography image utilizing the attenuation map to generate an attenuation-corrected positron emission tomography image (block 184). The method 156 further includes combining the computed tomography image with the attenuation-corrected positron emission tomography image to generate a motion artifact-free, motion-free combined image (e.g., diagnostic) of the region of interest (block 186). In particular, the computed tomography image is overlaid on the attenuation corrected positron emission tomography image to generate the motion artifact-free, motion-free combined image.

In certain embodiments, the method 156 may be modified to be utilized with cardiac phase matching instead of respiration phase matching. For example, the computed tomography scan data may be acquired at a determined cardiac phase of the cardiac cycle and the positron emission tomography scan data acquired over multiple cardiac cycles. Also, the positron emission tomography scan data may be gated (e.g., cardiac gated) into different cardiac phases. Further, a positron emission tomography image may be reconstructed only utilizing the positron emission tomography scan data acquired at a same cardiac phase as the determined cardiac phase that a computed tomography image was acquired.

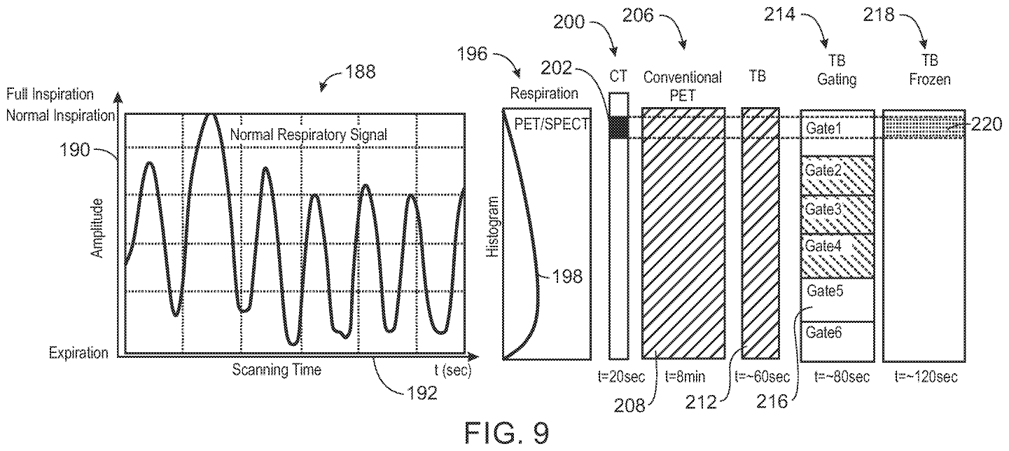

FIG. 9 is a schematic diagram illustrating the acquisition of data utilizing different types of positron emission tomography data acquisition relative to a respiratory cycle. Graph 188 depicts lung volume (e.g., amplitude) versus time (e.g., scanning time). Y-axis 190 represents amplitude and X-axis 192 represents scanning time. Plot 194 represents a normal respiratory signal. The upper portion of the Y-axis 190 indicates full or normal inspiration while the lower portion of the Y-axis 190 indicates expiration. Graph 196 depicts a histogram for positron emission tomography data of time spent in each phase. The histogram corresponds to inspiration and expiration in the graph 188. Area 198 of the histogram represents data in a period of minimal respiratory motion.

Schematic 200 represents the acquisition of the computed tomography data at a particular respiratory phase of the respiratory cycle as indicated by reference numeral 202. The computed tomography scan had a duration of 20 seconds. Dashed lines 204 indicates the corresponding region for positron emission tomography data in the schematics 206, 210, 214, and 218 to the computed tomography data 202 acquired at the particular respiratory phase. Schematic 206 represents the acquisition of positron emission tomography data with a conventional positron emission tomography (e.g., PET) scanner. The scan with the conventional positron emission tomography scanner had a duration of 8 minutes. As depicted in the schematic 206, the positron emission tomography data 208 is acquired for all of the phases of the respiratory cycle. Schematic 212 represents the acquisition of positron emission tomography data with a positron (large axial FOV) emission tomography scanner with no gating. The scan with the positron emission tomography scanner had a duration of 60 seconds. As depicted in the schematic 212, the positron emission tomography data 212 is acquired for all of the phases of the respiratory cycle. Schematic 214 represents the acquisition of positron emission tomography data with a positron emission tomography scanner where only the data acquired during a period of minimum motion (as indicated by reference numeral 216), via gating, is kept. The scan with the positron emission tomography scanner where only the data in the period of minimum motion is kept had a duration of 80 seconds. As indicated in schematic 214, the data 216 from the period of minimum motion does not match respiration phase of the acquired CT data 202. Schematic 218 represents the acquisition of positron emission tomography data with a positron emission tomography scanner where only the data (represented by reference numeral 220) that matches the respiratory phase of the acquired CT data 202 is kept in accordance with the method 88 in FIG. 5. The scan with the positron emission tomography scanner where only the data that matches the phase of the acquired CT data is kept had a duration of 1.5 minutes. Acquiring the positron emission tomography scan data over multiple respiratory cycles and over this scan duration enables a portion of the positron emission tomography data to be discarded (if needed) yet provide enough data for sufficient positron emission tomography imaging quality.

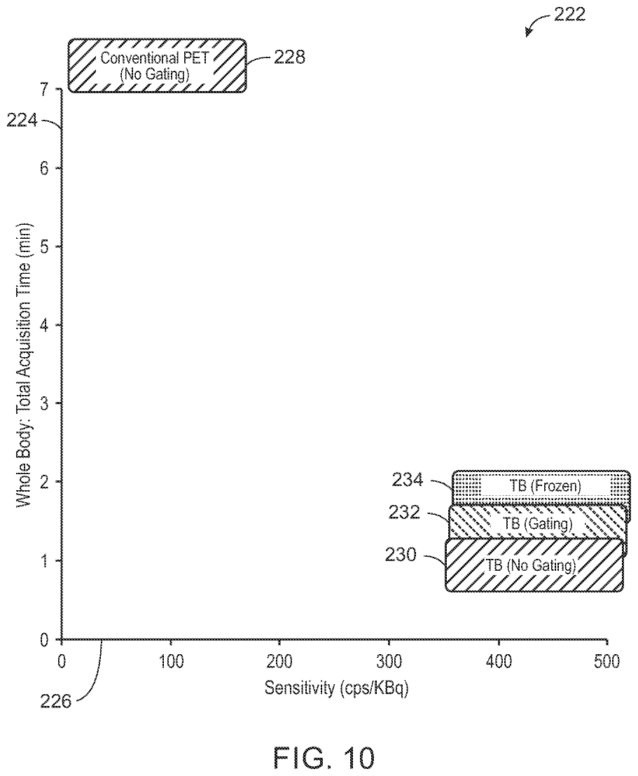

FIG. 10 is a graph 222 comparing acquisition time and sensitivity for different types of positron emission tomography data acquisition. Y-axis 224 represents total acquisition time to acquire data for the whole body of the subject. X-axis 226 represents the sensitivity. Plot 228 represents acquisition of positron emission tomography data with a conventional positron emission tomography (e.g., PET) scanner with no gating. Plot 230 represents acquisition of positron emission tomography data with a positron (large axial FOV) emission tomography scanner with no gating. Plot 232 represents acquisition of positron emission tomography data with a positron (large axial FOV) emission tomography scanner with gating. Plot 234 represents acquisition of positron emission tomography data with a positron (large axial FOV) emission tomography scanner where only positron emission tomography data that matches a phase (e.g., respiratory) of the acquired CT data is kept (e.g., called frozen). As depicted in the graph 222, acquiring data with a conventional positron emission tomography scanner takes significantly more time and is significantly less sensitive than acquiring data with a positron emission tomography scanner. Also, as depicted in the graph 222, acquiring positron emission tomography data with a positron (large axial FOV) emission tomography scanner where only positron emission tomography data that matches a phase (e.g., respiratory) of the acquired CT data is kept takes a little longer than the other techniques involving data acquisition with the positron emission tomography scanner to enable a portion of the positron emission tomography data to be discarded (if needed) yet provide enough data for sufficient positron emission tomography imaging quality.

Attenuation-corrected emission tomography images of a region of interest of a subject are displayed to the user for evaluation. Positron emission tomography scan data for the image with no gating may show blurring due to motion. Attenuation-corrected positron emission tomography scans acquired with a positron (large axial FOV) emission tomography scanner with gating are generally free of the blurring due to motion, but may show artifacts caused by inaccurate overlap of the emission image and the attenuation map. Attenuation-corrected emission tomography images, which are acquired with a positron (large axial FOV) emission tomography scanner where only positron emission tomography data that matches a phase (e.g., respiratory) of the acquired CT data is kept, may be free of both motion caused blurring and attenuation correction artifact.

Technical effects of the disclosed embodiments include providing systems and methods that utilize positron emission tomography (e.g., PET) photons that are acquired at the exact respiratory phase that the CT data is acquired to generate a positron emission tomography image. Technical effects of the disclosed embodiments include optimizing the combining of PET and CT imaging data (e.g., due to matching data acquired at a same respiration phase). Technical effects of the disclosed embodiments also include improving the image quality derived from the PET data due to improved attenuation correction. Technical effects of the disclosed embodiments further include improving diagnosis due to the full optimized combining of the PET and CT imaging data.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for combining computed tomography (CT) and positron emission tomography (PET) imaging data, comprising:
reconstructing, via a processor, a CT image of a region of interest of a subject based on CT scan data acquired at a determined respiratory phase with a CT scanner of a PET-CT imaging system;
generating, via the processor, an attenuation map from the CT image;
obtaining, via the processor, PET scan data of the region of interest of the subject acquired over multiple respiratory cycles with a PET scanner of the PET-CT imaging system;
gating, via the processor, the PET scan data into different respiratory phases; and
reconstructing, via the processor, a PET image utilizing only the PET scan data acquired at a same respiratory phase as the determined respiratory phase that the CT image was acquired and the attenuation map from the CT image.

2. The computer-implemented method of claim 1, further comprising:
performing, via the processor, attenuation correction on the PET image utilizing the attenuation map to generate an attenuation-corrected PET image; and
combining, via the processor, the CT image with the attenuation-corrected PET image to generate a motion artifact-free, motion-free combined image of the region of interest.

3. The computer-implemented method of claim 1, further comprising, prior to reconstructing the PET image, determining, via the processor, from the PET scan data only the PET scan data acquired at the same respiratory phase as the determined respiratory phase that the CT image was acquired utilizing a list mode file of the PET scan data.

4. The computer-implemented method of claim 1, wherein reconstructing the PET image comprises:
reconstructing, via the processor, a plurality of PET frames from the PET scan data; and
determining, via the processor, which PET frames of the plurality of PET frames were acquired at the same respiratory phase as the determined respiratory phase that the CT image was acquired to utilize as the PET image.

5. The computer-implemented method of claim 4, wherein each frame of the plurality of PET frames spans less than 25 percent of a mean respiratory cycle.

6. The computer-implemented method of claim 4, wherein the PET image is reconstructed from only the PET frames of the plurality of PET frames that were acquired at the same respiratory phase as the determined respiratory phase that the CT image was acquired.

7. The computer-implemented method of claim 1, further comprising, prior to obtaining the PET scan data, obtaining, via the processor, initial PET scan data acquired with the PET scanner simultaneously with the acquisition of the CT scan data acquired at the determined respiratory phase with the CT scanner so that the initial PET scan data and the CT scan data are acquired at a same respiratory phase.

8. The computer-implemented method of claim 7, further comprising, prior to reconstructing the PET image, determining, via the processor, from the PET scan data only the PET scan data from a list mode file of the PET scan data that matches the initial PET scan data.

9. The computer-implemented method of claim 7, wherein reconstructing the PET image comprises:
reconstructing, via the processor, a plurality of PET frames from the PET scan data, wherein each frame of the plurality of PET frames spans less than 25 percent of a mean respiratory cycle; and
determining, via the processor, which PET frames of the plurality of PET frames that match the initial PET scan data to utilize as the PET image, wherein the PET image is reconstructed from only the PET frames of the plurality of PET frames that match the initial PET scan data.

10. A system for combining computed tomography (CT) and positron emission tomography (PET) imaging data, comprising:
a memory encoding processor-executable routines;
a processor configured to access the memory and to execute the processor-executable routines, wherein the processor-executable routines, when executed by the processor, cause the processor to:
reconstruct a CT image of a region of interest of a subject based on CT scan data acquired at a determined respiratory phase with a CT scanner of a PET-CT imaging system;
obtain PET scan data of the region of interest of the subject acquired over multiple respiratory cycles with a PET scanner of the PET-CT imaging system;
gate the PET scan data into different respiratory phases;
reconstruct a PET image utilizing only the PET scan data acquired at a same respiratory phase as the determined respiratory phase that the CT image was acquired;
generate an attenuation map from the CT image;
perform attenuation correction on the PET image utilizing the attenuation map to generate an attenuation-corrected PET image; and
combining the CT image with the attenuation-corrected PET image to generate a motion artifact-free, motion-free combined image of the region of interest.

11. The system of claim 10, wherein the processor-executable routines, when executed by the processor, further cause the processor, prior to reconstructing the PET image, to determine from the PET scan data only the PET scan data acquired at the same respiratory phase as the determined respiratory phase that the CT image was acquired utilizing a list mode file of the PET scan data.

12. The system of claim 10, wherein the processor-executable routines, when executed by the processor, further cause the processor to:
reconstruct a plurality of PET frames from the PET scan data, wherein each frame of the plurality of PET frames spans less than 25 percent of a mean respiratory cycle; and
determine which PET frames of the plurality of PET frames were acquired at the same respiratory phase as the determined respiratory phase that the CT image was acquired to utilize as the PET image.

13. The system of claim 12, wherein the PET image is reconstructed from only the PET frames of the plurality of PET frames that were acquired at the same respiratory phase as the determined respiratory phase that the CT image was acquired.

14. The system of claim 10, wherein the processor-executable routines, when executed by the processor, further cause the processor, prior to obtaining the PET scan data, to obtain initial PET scan data acquired with the PET scanner simultaneously with the acquisition of the CT scan data acquired at the determined respiratory phase with the CT scanner so that the initial PET scan data and the CT scan data are acquired at a same respiratory phase.

15. The system of claim 14, wherein the processor-executable routines, when executed by the processor, further cause the processor, prior to reconstructing the PET image, to determine from the PET scan data only the PET scan data from a list mode file of the PET scan data that matches the initial PET scan data.

16. The system of claim 14, wherein the processor-executable routines, when executed by the processor, further cause the processor, prior to reconstructing the PET image, to:
reconstruct a plurality of PET frames from the PET scan data, wherein each frame of the plurality of PET frames spans less than 25 percent of a mean respiratory cycle; and
determine which PET frames of the plurality of PET frames that match the initial PET scan data, wherein the PET image is reconstructed from only the PET frames of the plurality of PET frames that match the initial PET scan data.

17. A non-transitory computer-readable medium, the computer-readable medium comprising processor-executable code that when executed by a processor, causes the processor to:
reconstruct a computed tomography (CT) image of a region of interest of a subject based on CT scan data acquired at a determined respiratory phase with a CT scanner of a positron emission tomography (PET)-CT imaging system;
obtain PET scan data of the region of interest of the subject acquired over multiple respiratory cycles with a PET scanner of the PET-CT imaging system;
gate the PET scan data into different respiratory phases;
reconstruct a PET image utilizing only the PET scan data acquired at a same respiratory phase as the determined respiratory phase that the CT image was acquired;
generate an attenuation map from the CT image;
perform attenuation correction on the PET image utilizing the attenuation map to generate an attenuation-corrected PET image; and
combine the CT image with the attenuation-corrected PET image to generate a motion artifact-free, motion-free combined image of the region of interest.

18. The non-transitory computer-readable medium of claim 17, wherein the processor-executable code, when executed by the processor, further cause the processor, prior to reconstructing the PET image, to determine from the PET scan data only the PET scan data acquired at the same respiratory phase as the determined respiratory phase that the CT image was acquired utilizing a list mode file of the PET scan data.

19. The non-transitory computer-readable medium of claim 17, wherein the processor-executable code, when executed by the processor, further cause the processor, prior to reconstructing the PET image, to:
reconstruct a plurality of PET frames from the PET scan data, wherein each frame of the plurality of PET frames spans less than 25 percent of a mean respiratory cycle; and
determine which PET frames of the plurality of PET frames were acquired at the same respiratory phase as the determined respiratory phase that the CT image was acquired, wherein the PET image is reconstructed from only the PET frames of the plurality of PET frames that were acquired at the same respiratory phase as the determined respiratory phase that the CT image was acquired.

20. The non-transitory computer-readable medium of claim 17, wherein the processor-executable code, when executed by the processor, further cause the processor, prior to obtaining the PET scan data, to obtain initial PET scan data acquired with the PET scanner simultaneously with the acquisition of the CT scan data acquired at the determined respiratory phase with the CT scanner so that the initial PET scan data and the CT scan data are acquired at a same respiratory phase.
